(19) United States
(12) Patent Application Publication
(10) Pub. No.: US 2011/0172620 A1
(43) Pub. Date: Jul. 14, 2011

(54) FEMALE URINARY AID

(75) Inventor: Yazdiyar Meherji Khambatta, Blockhouse Bay (NZ)

(73) Assignee: IP ON DEMAND, Parnell, Auckland (NZ)

(21) Appl. No.: 13/062,468

(22) PCT Filed: Sep. 7, 2009
(86) PCT No.: PCT/NZ09/00189

§ 371 Date: Mar. 4, 2011

(30) Foreign Application Priority Data
Sep. 5, 2008 (NZ) 571020

(51) Int. Cl. A61F 5/455; B23P 17/04

(52) U.S. Cl. 604/347; 29/428

(57) ABSTRACT

An urinary aid able to be positioned by a female user to allow that female user to urinate with control, the device comprising a tube-like engagement head having a urine receivable end for engaging the mouth of the urethra and a urine discharge end, tube-like engagement head comprising a locator element, located at the urine receivable end of the engagement head that allows urine from the female to access the urine discharge end of the engagement head, the locator element extending substantially transverse to the walls of the tube-like engagement head to define (a) an inner locator element region that extends from about the wall of the tube-like engagement head towards the centre of the tube-like engagement head, and (b) an outer locator element region, that extends radially outwards from the wall of the tube-like engagement head, and a conformable material attached to, or formed unitarily with, the locator element, such that positioning of the locator element by the user against the urethral opening actuates the locator element to move into a substantially sealing configuration about the users urethral opening.

### FIELD OF THE INVENTION

The present invention relates to an urinary aid for woman. More particularly but not exclusively it relates to an urinary aid that operatively fits within the folds of the labia minora.

### BACKGROUND OF INVENTION

The use of public toilets can be an unwelcome experience for females, both in terms of hygiene and their requirement to squat over the toilet. Some contactable infections and venal diseases can be transmitted through contact with a toilet seat.

Additionally, urinating outside (i.e. when hiking, camping or travelling) can also be inconvenient.

Various urinary aids are known. For example, US patent application 20080262448 of Padmanabhan Mahalingam describes a device consisting of a cup that fits about the urethra for collecting urine, a shield, and an outlet tube. US patent application 20050097662 of Brett Leimkuhler describes a device having a funnel and a flexible tube, the funnel contoured to conform to the labia region. U.S. Pat. No. 5,893,176 of Rainbow Medical describes a device that has two sealing rings, the interior ring comprising a circumferential wall that engages the exterior of the labia minor and an exterior ring that engages the exterior of the labia majora. US patent application 20090089919 of Cynthia Rudolph describes a biodegradable funnel-shaped female urination device.

Problems with such devices include
a propensity to leak,
uncomfortable to use,
not convenient (in terms of carrying by a female), and
not easily disposable.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### OBJECT OF THE INVENTION

It is an object of the present invention to provide an urinary aid for woman that overcomes or at least ameliorates some of the abovementioned disadvantages or which at least provides the public with a useful choice.

Other objects of the invention may become apparent from the following description which is given by way of example only.

### SUMMARY OF THE INVENTION

Broadly stated, the invention relates to a tube-like device for sealing against a fluid opening, the device comprising a locator element that extends substantially transverse to the walls of the tube-like device, with an aperture therein to allow fluid flow, having (a) an inner region that extends from about the wall of the tube-like device head towards the centre of the tube-like device and (b) an outer region, that extends radially outwards from the wall of the tube-like device,

such that positioning of the locator element by the user against a fluid opening actuates the locator element through deflection of the inner region away from the user and simultaneous deflection of the outer region towards the fluid opening to form a seal.

Another aspect of the invention relates to an urinary aid able to be positioned by a female user to allow that female user to urinate with control, the device comprising:
(A) a tube-like engagement head having a urine receivable end for engaging the mouth of the urethra and a urine discharge end, the tube-like engagement head comprising
   (i) a locator element, located at the urine receivable end of the engagement head that allows urine from the female to access the urine discharge end of the engagement head, the locator element extending substantially transverse to the walls of the tube-like engagement head to define (a) an inner locator element region that extends from about the wall of the tube-like engagement head towards the centre of the tube-like engagement head, and (b) an outer locator element region, that extends radially outwards from the wall of the tube-like engagement head, and
   (ii) a conformable material attached to, or formed unitarily with, the locator element,

such that positioning of the locator element by the user against the urethral opening actuates the locator element to move into a substantially sealing configuration about the users urethral opening.

In some embodiments the positioning of the locator element against the urethral opening by the user actuates the inner locator element such that the outer locator element moves towards a substantially urethral opening sealing configuration.

In some embodiments the inner locator element is deflected inward of the tube-like engagement head such that the outer locator element is simultaneously deflected or moved towards or about the urethral opening of the user or into a substantially sealing configuration.

In some embodiments, when in use, positioning of the locator element against the urethral opening causes the locator element to act as a fulcrum as the urethral opening pushes against the inner region causing the outer region to move towards the female user, resulting in a seal being formed between the user and the urinary aid.

Another aspect of the invention relates to an urinary aid able to be positioned by a female user to assist that female to urinate with control, the device comprising

an engagement head that engages the urine receivable end of the ducting apparatus,

wherein the engagement head includes an annular seal member about an entrance of the engagement head, which entrance can allow urine to the ducting apparatus, and

wherein variation of the disposition of the ducting apparatus to at least part of the engagement head or to the annular seal, or both, can create a couple on the annular section thereby to vary conformation of the annular seal member to that female.

Another aspect of the invention relates to an urinary aid able to be positioned by a female user to assist that female to urinate with control, the device comprising

a first annular seal located about the urine receivable end of the duct, the first annular seal adapted to seal against the urethra and be contained within the labia minora,

a second annular seal located radially outwards of the first annular shield, the second annular shield adapted to seal against the user, and be contained within the labia minora.

The following embodiments may relate to any of the above aspects.

In some embodiments the urinary device is formed with a duct, or the duct is applied to the device just prior to use.

In some embodiments the ducting is attached to the urinary aid just prior to use.

Another aspect of the invention relates to a pack of urinary aids of the present invention, with or without the ducting.

In some embodiments the duct is a flexible hose. Preferably the duct includes an expanding concertina arrangement, or bellows that provide for its bendability.

In some embodiments the duct is telescopic.

In some embodiments the locator element is a contoured circular seal adapted to form a seal when pressed around the mouth of urethra opening.

In some embodiments the locator element self aligns to the mouth of the urethra to form the seal. Preferably the locator element is moveable to facilitate the sealing of the locator element to the mouth of the urethra.

In some embodiments the conformable material is present as a layer of soft absorbent material on at least part of the locator element. Preferably the conformable material is present on that part of the locator element that forms a seal against the user.

In an alternate embodiment the conformable material is present about the locator element.

In some embodiments the locator element and the conformable material are formed from a single material. Preferably the material for the conformable material remains soft while the material for the locator element is compressed so as to have an increased rigidity compared to the conformable material.

In an alternate embodiment the locator element and the conformable material are formed from two or more different materials, joined to each other by gluing, heat adhering, stitching, or any other method for attaching a soft and absorbable material to a more rigid material.

In some embodiment the seal between the user and the urinary aid is facilitated by hydration of one or both of the locator element or the conformable material.

In some embodiments the urinary device comprises one or more sealing rings radially outwards of the locator element.

In some embodiments the urinary device comprises an exterior shell. Preferably the shell is formed from a plastic or a rigid paperboard material. Preferably the shell is biodegradable.

In some embodiments the locator element and/or conformable material are coated or imbibed with a lubricant, an anti-bacterial or a perfume, scent or antifungal or a combination thereof.

In some embodiments at least part thereof of the urinary aid is biodegradable. Preferably the locator element is biodegradable. Preferably the locator element and the conformable material are both biodegradable. Preferably the locator element, the conformable material and the duct are all biodegradable. Preferably the conformable material is biodegradable. Preferably the conformable material and the duct are both biodegradable. More preferably the entire urinary device is biodegradable.

In some embodiments the locator element is formed, at least in part by, wood pulp, tissue, polyacrylate polymers, absorbing fabric, absorbing paper, gelatine, starch, cellulose, alginate or carageenan, or a combination thereof.

In some embodiments the absorbable material is formed, at least in part by, wood pulp, tissue, polyacrylate polymers, absorbing fabric, absorbing paper, gelatine, starch, cellulose, alginate or carageenan, or a combination thereof.

In some embodiments the duct comprises a one way valve. Preferably the valve opens in response to the collection of a small amount of urine. More preferably the valve only opens when about 0.1, 1, 2, 3, 4, 5, 6, 7, 8, 9, 10, 11, 12, 13, 14, 15, 16, 17, 18, 19, 20, 25, 30, 35, 40, 45, or 50 ml of urine is accumulated.

In some embodiments the engagement head is 10, 15, 20, 25, 30, 35, 40, 45 and 50 millimetres in diameter. More preferably the engagement head is about 25 millimetres in diameter.

In some embodiments the urinary aid comprises an air excluder, aperture or inlet to allow air flow between the environment and the internal ducting of the urinary aid so that when in use (and the aid is sealed against the user) air can pass out/in of the device (without needing to pass through the distal opening of the duct) to avoid a vacuum forming.

In some embodiments the urinary aid 1 is positioned against the urethral opening and more preferably against the urethral meatus.

Another aspect of the invention relates to a method of making an urinary aid of the present invention comprising the steps (in no particular order) of:

forming the locater element, wherein the locator element is adapted to seal about the mouth of the urethra,

forming the absorbable material on or about the locator element,

attaching a duct to the locator element and absorbable material pair,

such that when in use the urine passes from the urethra past the locator element and into the duct.

In some embodiments the locator element is formed unitarily with the absorbable material. Preferably the locator element material is compressed to form it as a rigid material. More preferably the locator element and absorbable material are compression moulded, with the locator element material having greater compression (and thus increased density) compared to the absorbable material.

In an alternate embodiment the locator element and the absorbable material are formed from separate materials. Preferable the locator element and the absorbable material are bonded to each other. In one embodiment the locator element and the absorbable material are heat moulded together, the heat moulding bonding the two materials together.

In one embodiment the method of forming the urinary aid comprises the step of forming the device into a lie flat blank, wherein the lie flat blank can be inflated or pulled into its 3-dimensional operational state.

In one embodiment the urinary aid is packaged in a compressed condition. Preferable the compressed urinary aid is bias to reform to its 3-dimensional condition.

Another aspect of the invention relates to a method of using an urinary aid comprising the steps of
1. obtaining an urinary aid that comprises
   (a) a duct having a urine receivable end a urine discharging end,
   (b) a locator element, located at the urine receivable end of the duct that allows urine from the female to access the duct, that effectively extends substantially transverse to the duct to define (a) an inner locator element region that extends from about the wall of the duct towards the notional centre of the duct, and (b) an outer locator element region, that extends away radially outwards from the wall of the duct, and
   (c) a conformable and absorbable material (“conformable material”) attached or formed unitarily with the locator element,
2. wherein the female user applies the device to the mouth of her urethra so that the locator element forms a seal substantially about the urethral mouth, such that the locator element is positioned within the confounds of the labia minora.

In some embodiments the use of the device includes the step of pressing the urinary aid against the mouth of the urethra so that the conformable material, located about the locator element, pushes forward in a fulcrum motion so that the conformable material forms a seal against the female user. Preferably the seal is formed within the confounds of the labia minora.

In one embodiment after use, the urinary aid is rotated slightly such that the conformable material can absorb any urine that has escaped the seal between the locator element and the user. This also aids as a wipe.

Another aspect of the invention relates to the use of an urinary aid of the present invention.

Another aspect of the invention relates to an urinary aid substantially described, with or without reference to any one or more of the accompanying drawings.

Other aspects of the invention may become apparent from the following description which is given by way of example only and with reference to the accompanying drawings.

As used herein the term “conformable” includes the ability to adopt a shape complementary to the object to which it is being applied.

As used herein the term “biodegradable” means that the material will decompose or break down when exposed to the environment. In some embodiments, the biodegradation occurs within two weeks, three weeks, one month, one, two, three, four, five, six, seven, eight, nine, ten, eleven or twelve months. More preferably, the biodegradation does not require any active steps on behalf of the user, the device fully breaking down when or after being disposed.

As used herein the term “and/or” means “and” or “or”, or both.

As used herein “(s)” following a noun means the plural and/or singular forms of the noun.

The term “comprising” as used in this specification and claims means “consisting at least in part of”. When interpreting statements in this specification and claims which include that term, the features, prefaced by that term in each statement, all need to be present but other features can also be present. Related terms such as “comprise” and “comprised” are to be interpreted in the same manner.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

### BRIEF DESCRIPTION OF THE DRAWINGS

The invention will now be described by way of example only and with reference to the drawings in which:

FIG. 1 is a section view through AA of FIG. 3C of an urinary aid of the present invention having the locator element adapted to seal against the mouth of the urethra,

FIG. 2 is a section view through AA of FIG. 3C showing the fulcrum like movement of the locator element of an urinary aid of FIG. 1,

FIG. 3A is a section view through AA of FIG. 3C showing the locator element adapted to seal against the mouth of the urethra,

FIG. 3B is a section view through AA of FIG. 3C showing the locator element adapted to seal against the mouth of the urethra with a layer of conformable material that forms a layer between the locator element and the female user,

FIG. 3C is an end view of the locator element and conformable material of an embodiment of the invention whereby the conformable material surrounds the locator element,

FIG. 4 shows a side view of a duct,

FIG. 5 shows a perspective view of an alternate embodiment of the invention, showing an urinary device with multiple annular seals,

FIG. 6 shows a cross-section view of an urinary aid of FIG. 5 with the duct attached,

FIG. 7 shows a cross-section view of an urinary aid of FIG. 5 without the duct attached,

FIG. 8A shows an end view of an urinary aid of FIG. 5,

FIG. 8B shows an end view of an urinary aid of FIG. 5,

FIG. 9 shows a cross-section view through BB of FIG. 12B,

FIGS. 10A and 10B show a end and side view of an urinary aid of FIG. 8,

FIG. 11A shows a locator element of an urinary aid of FIG. 8,

FIG. 11B shows an end view of a locator element of FIG. 8,

FIG. 12 shows the use of an urinary device of FIG. 1,

FIG. 13 shows the use of an urinary device of FIG. 9,

FIG. 14 shows how an urinary aid of the present invention fits to the female anatomy,

FIG. 15 shows urinary devices of the present invention in the form of a pen clip,

FIGS. 16 and 17 show how multiple urinary aids can be stored in an indexed manner when present without the duct,

FIG. 18 show how urinary device of the present invention without the duct can be stored in a container, and

FIG. 19 shows how urinary device of the present invention without the duct can be stored in a container for ease of use.

### DETAIL DESCRIPTION OF THE INVENTION

The invention can broadly be stated to relate to a small portable urinary aid that can be held by a female against their urethral opening, to permit them to urinate with control. For example, such an urinary aid will allow the female to urinate while crouched, standing, sitting or leaning against a support.

The urinary aid comprises:
an engagement head 2 formed of a locator element 3 and an conformable material attached or formed unitarily with the locator element 3,
a duct 4 (that may be supplied with the urinary aid or supplied separately and attached to the urinary aid just prior to use), and
optionally a shell 11.

The urinary device 1 is formed from an engagement head 2 that is attached to a duct 5 whereby the engagement head is placed against the mouth of the urethral opening to form a seal. Preferably there is located on or about the locator element 3 conformable material 4. The conformable material 4 can form a layer on top of the locator element 3 so that when placed against the female user it forms a soft layer between the user and the locator element 3. In addition, or alternatively, the conformable material 4 can be located radially outwards and around the locator element 3.

Preferably the conformable material 4 is of a lower density than the locator element 3. In these embodiments the locator element 3 is more rigid than the conformable material 4 and may also have a higher density. The conformable material 4 is preferably soft and absorbable so as to be comfortable for the user and to prevent seepage of any urine that escapes during use.

In some embodiments, the duct includes a one-way valve. The valve is preferably designed to open in response to the accumulation of a certain amount of urine. The purpose of such a valve is to prevent dribbles emanating from the urine discharge end of the duct which may mark the user's clothing. The valve collects that small amount of dribble and prevents it from dribbling out of the end of the duct. Preferably the valve only opens when about 1, 2, 3, 4, 5, 6, 7, 8, 9, 10, 11, 12, 13, 14, 15, 16, 17, 18, 19, 20, 25, 30, 35, 40, 45 or 50 ml of urine is accumulated, and useful ranges may be selected between any of these values (these values for example, about 1 to about 50, about 1 to about 40, about 7 to about 47, about 12 to about 50, about 17 to about 24, about 21 to about 49, about 26 to about 43, about 31 to about 50, about 38 to about 46, about 41 to about 48).

When sealed against the user and the duct 5 having been filled with urine, a vacuum may form. In some embodiments, the urinary aid also comprises an air-excluder, aperture or inlet that allows air flow between the environment and the internal ducting of the urinary aid 1 so that when in use, and the aid is sealed against the user, air can pass in or out of the urinary aid, without needing to pass through the urine outlet end of the duct, to avoid a vacuum forming.

In one embodiment, the urinary aid is comprised of engagement head 2 that comprises at the urine receivable end 6 a number of concentric seals concentrically disposed from each other. Preferably the first seal is disposed concentrically around the urine receivable end 6 of the engaging head 2 and is adapted to fit conformably about the mouth of the urethral opening. As seen in FIG. 5, a device may include multiple seals including an inner seal 13 and an outer seal 14. As further seen in FIG. 5, the outer seal may be raised in relation to the inner seal.

In some embodiments the device may comprise multiple seals, each seal being disposed concentrically and radially outwards of the inner seal. Each seal helps reduce the potential for urine leakage to occur.

An advantage of the urinary aid 1 of the present invention is that it is of a very small size.

In some embodiments the size of the engagement head is 10, 15, 20, 25, 30, 35, 40, 45 and 50 millimetres, and useful ranges may be selected from any of these values (for example, about 1 to about 40, about 7 to about 47, about 12 to about 50, about 17 to about 24, about 21 to about 49, about 23 to about 47, about 26 to about 43, about 28 to about 43, about 31 to about 50, about 35 to about 41, about 38 to about 46, about 41 to about 48).

Preferably the thickness of the engagement head is approximately 5, 10, 15, 20, 25, 30, 35, 40, 45 and 50 millimetres, and useful ranges may be selected between any of these values (for example, about 1 to about 40, about 7 to about 47, about 12 to about 50, about 17 to about 24, about 21 to about 49, about 23 to about 47, about 26 to about 43, about 28 to about 43, about 31 to about 50, about 35 to about 41, about 38 to about 46, about 41 to about 48).

### 1. Engagement Head

The engagement head 2 engages to the mouth of the urethral opening to form a seal. The engagement head 2 has an essentially tube-like arrangement to allow the passage the urine from the urine receivable end 6 to the urine discharge end 7 of the engagement head 2.

In some embodiments the engagement head 2 can be sold separately to a duct 5, the urinary aid 1 being capable of assembly by attaching the duct 5 to the urine dischargeable end 7 of the engagement head 2.

At the urine receivable end 6 of the engagement head 2 there is present a locator element 3 that extends transversally to the walls of the engagement head 2. The locator element 3 comprises an aperture to allow flow of urine through the engagement head 2 to the urine discharge end 7 of the engagement head 2.

The engagement head 2 also includes the conformable material 4, that can be formed either as a layer on top of the locater element 3 or formed radially about the locator element.

The engagement head 2 may also include a liquid impermeable shell 11.

### 2. Locator Element

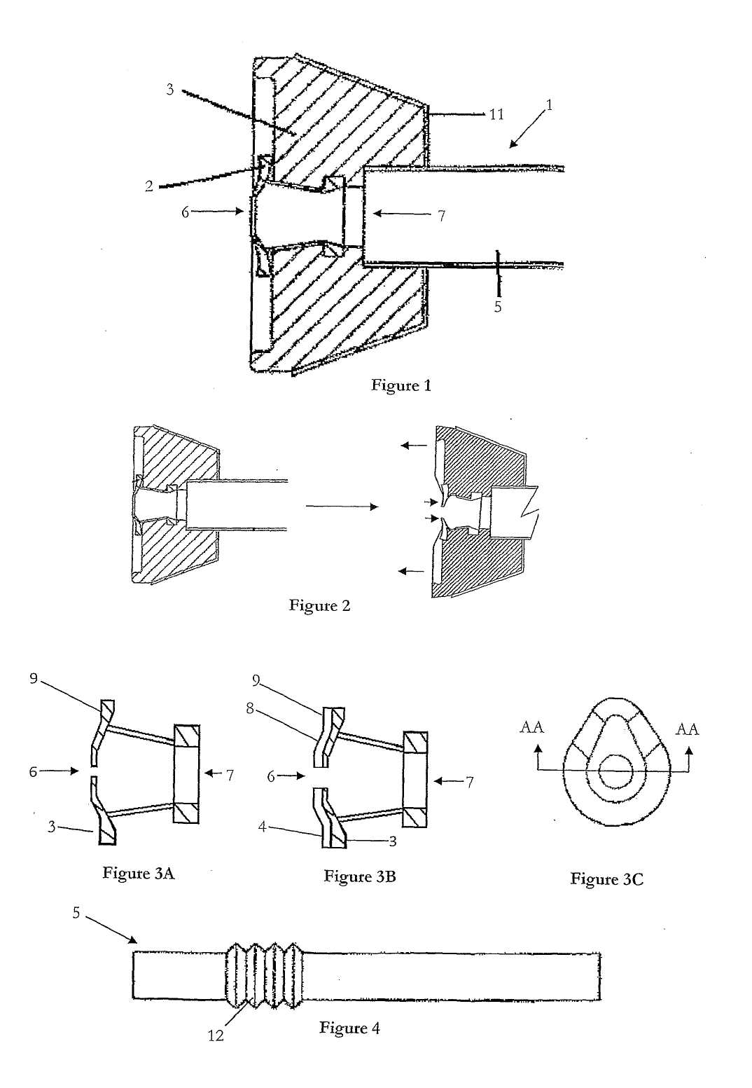

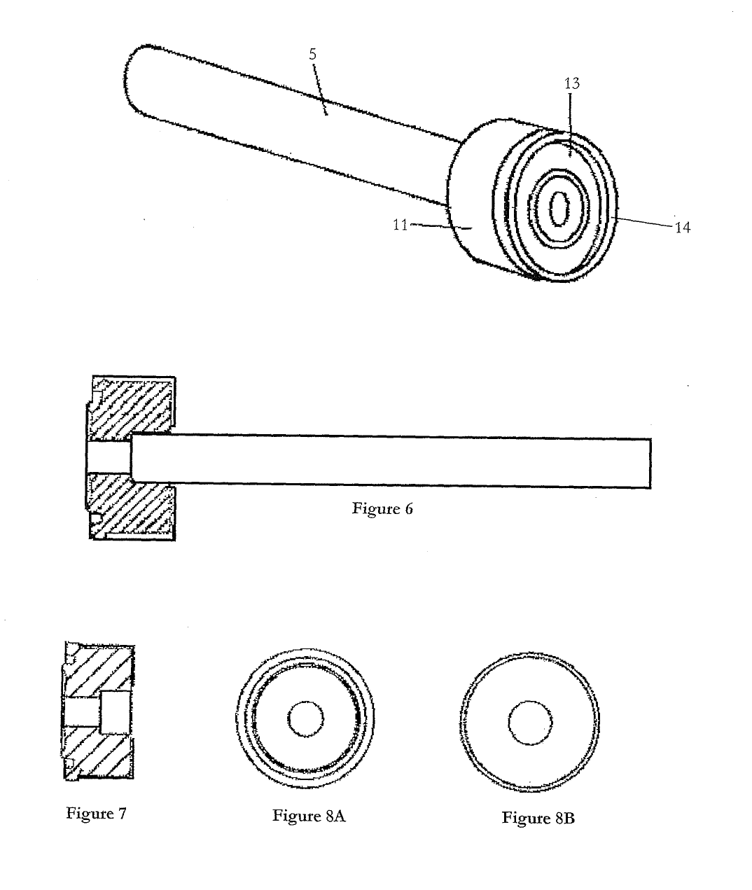

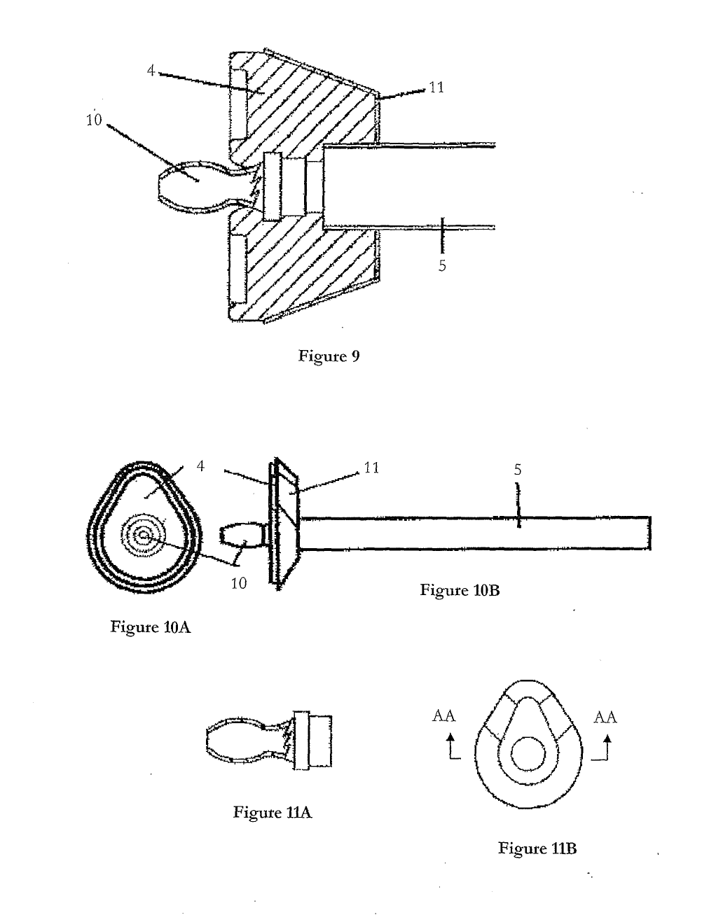

FIG. 1 shows an urinary aid 1 with the locator element 2 shown in greater detail in the cross-section in FIGS. 2 and 3. The locator element 3 helps to locate the urinary aid 1 to the mouth of the urethral opening to form a seal between the urinary aid 1 and the user.

A feature of the locator element 3 is its ability to “self align” to the mouth of the urethral opening which helps form a seal and prevent leakage of the urine. More preferably the locator element 3 of the urinal aid 1 aligns against the urethral meatus.

It should be appreciated that the locator element 3 and the conformable material 4 of the engagement head 2 may be manufactured as one piece, for example, through plastic moulding. Alternatively the locator element 3 and the conformable material 4 of the engagement head 2 could be formed as separate pieces which are then bonded to each other.

As seen in detail in the cross-section shown in FIGS. 2 and 3, the locator element 3 is positioned substantially transverse to the walls of locator element 3. The locator element 3 has an inner region 8 and an outer region 9. The inner region 8 of the locator element 3 is located radially inwards of the walls of the engagement head 2 and the outer region 9 of locator element 3 is located radially outwards of the walls of the engagement head 2.

The operation of the locator element 3 is shown in FIG. 2 by the arrows. Force applied to the inner region 8 of the locator element 3 causes the inner region 8 to be deflected inwardly to the engagement head 2 causing a simultaneous deflection of the outer region 9 of the locator element 3 towards the user. This actuation of the locator element 3 is similar to a fulcrum action with the axis of rotation being about where the wall of the engagement head 2 meets the locator element 3.

As mentioned above, in some embodiments the locator element 3 is a rigid material, as a too soft material would merely deflect without leading to a simultaneous deflection of the outer region 9.

In some embodiments the locator element 3 locates solely on the mouth of the urethral opening. The mouth of the urethra is surrounded by a raised circular portion (like the mouth of a volcano). The vaginal opening is located below the urethral opening and both openings are surrounded by the labia minora. The whole region is surrounded by the labia majora. The urinary device 1, when in use, is centred upon the mouth of the urethra opening and the raised surrounding portion of the urethra and placed such that the engagement head 2 lies within the folds of the labia minora.

One advantage of the locator element is that it helps locate the device onto the mouth of the urethral opening.

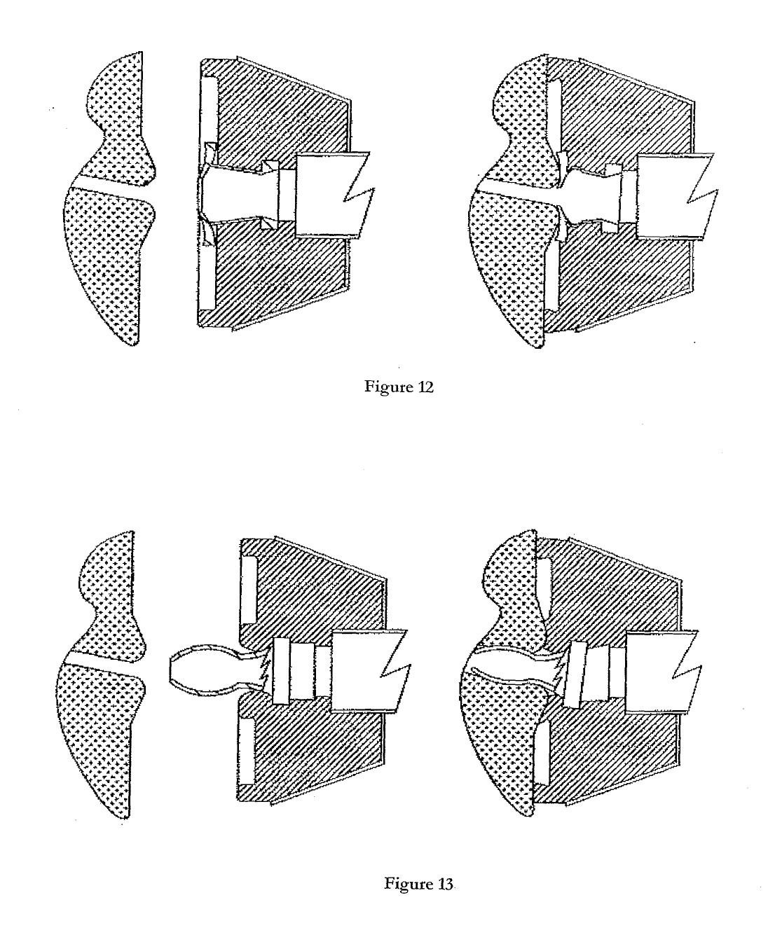

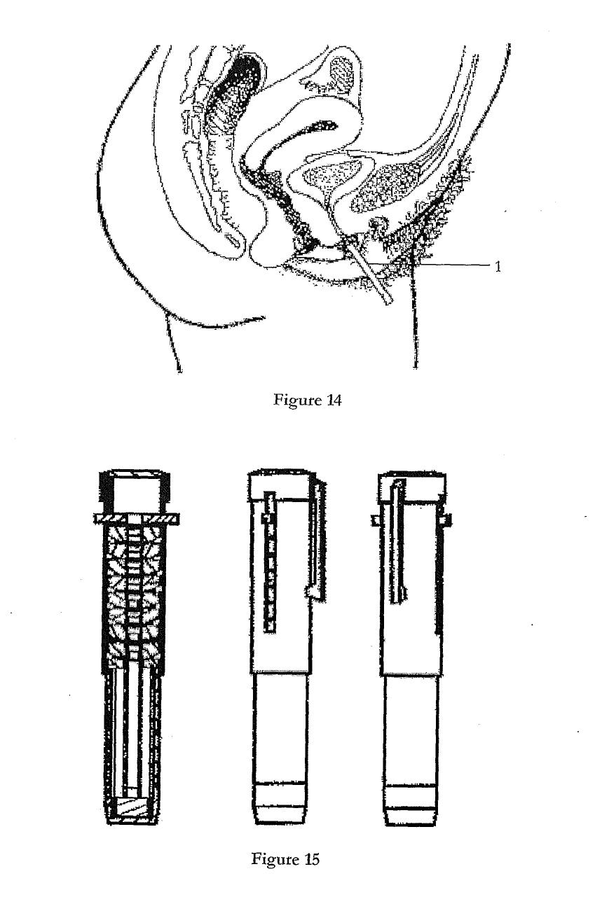

The locator element may have a projection 10 as seen in FIGS. 9 and 10. The projection 10 inserts into the urethra, the projection 10 having smooth round edges and is small enough to centre on the urethral opening. For example, the projection 10 may be a bulging elliptical section or a cylindrical tube. Typically the entry is only a few millimetres into the mouth of the urethra, thus causes minimal or no discomfort to the user.

As described above, in other embodiments the locator element is a soft seal that presses around the mouth of the urethra avoiding the need to insert it into the urethral opening.

### 3. Conformable Material

The engagement head 2 includes conformable material 4, for enhancing the comfort of the device in use and helping prevent leakage of any urine.

In some embodiments the conformable material 4 is compressible. Preferably the conformable material is also absorbable.

In some embodiments the conformable material 4 is made of a fibrous material such as used in nappies. It should be appreciated that other material could be used such as wood pulp (that is highly absorbent), polyacrylate polymers (a highly absorbent material in powdered form) that may be packed inside a layer of wood pulp an absorbing fabric, an absorbing paper, gelatin, starch, cellulose, alginate or carageenan, or a combination thereof.

As seen in FIGS. 1 and 2 the conformable material 4 may be formed radially outwards of the locator element 3 so that when in use, the conformable material 4 is displaced forwards and forms a seal against the user.

As further shown in FIGS. 1 and 2 the conformable material 4 can be manufactured to surround the entirety of the engagement head 2.

In some embodiments the locator element 3 and conformable material 4 are formed from two different materials, the locator element 3 being formed from a material with a higher rigidity than the conformable material 4. In some embodiments, or additionally, the conformable material 4 has bonded to the locator element 3. This bonding can be in the form of gluing, heat compression, or any other method known in the art.

As shown in FIG. 3B the conformable material 4 may be formed as a layer on top of the locator element 3. In such embodiments, when the engagement head 2 is placed against the urethral opening the conformable material 4 is pressed against the user. The locator element 3 still actuates since the mouth of the urethral opening presses against the inner region 8 of the locator element 3 pushing the outer region 9 of the locator element 3 against the user.

The conformable material may be formed as a layer along the outer region of the locator element 3. In this form the locator element will position against the urethral opening and the conformable material 4 will also form a seal adjacent the locator element 3.

In some embodiments the locator element 3 and the conformable material 4 are formed from a single material. For example, the locator element 3 and the conformable material 4 may be formed from a plastic such as polystyrene or polyurethane. The increased rigidity of the locator element 3 compared to the conformable material 4 can be provided for through differential compression of the locator element 3 and the conformable material. For example, to obtain a locator element 3 that has a higher rigidity than the conformable material 4 the material that is to form the locator element 3 can be subjected to a higher compression than the material that is to form the conformable material 4. Such manufacturing will result in a locator element 3 having a greater density and rigidity in comparison to the conformable material 4.

In some embodiments the surface of the locator element 3, conformable material 4 or the entire engagement head 2 is coated or imbibed with an antibacterial or antiseptic film for hygiene and comfort. It should be appreciated that other additives such as scent could be coated to or imbibed into the locator element 3, and/or the conformable material 4 of the engagement head 2.

### 4. Shell

In some embodiments the urinary aid 1 comprises a shell 11 that encapsulates the lower portion of the engagement head 2. The function of the shell 11 is to hold and contain the engagement head 2 (that comprises the locator element 3 and the conformable material 4). Preferably the shell 11 is impervious to liquid and thus provides an impenetrable membrane that liquid cannot pass through. This will prevent any leakage of urine outside of the urinary aid 1.

It should be appreciated that the shape of the shell 11 conforms to the engagement head 2 of the urinary aid 1. For example, in those embodiments where the conformable material 4 is located about the locator element 3 the shell 11 will be formed about the conformable material 4.

In those embodiments where the conformable material 4 is formed as a layer on top of the locator element 3 then the shell 11 may form a layer about the engagement head 2 up to the edge of the locator element 3.

The shell 11 can be formed from any impermeable material such as a plastic or coated fibreboard. For example, some fibreboard has a coating that makes it impervious and/or less impervious to liquid. In some instances this may be a plastic coating.

### 5. Duct

The urinary aid 1 comprises a duct 5 that attaches to, or within, the urine discharge end 7 of the engagement head 2.

The purpose of the duct 5 is to direct urine away from the engagement head 2.

It should be appreciated that the length of the duct 5 can be at any length and can include any number of the different modifications such as a flexible bellow 12 (as seen in FIG. 4) or it could be formed telescopically. The advantage of a telescopic duct is that it makes the urinary aid 1 more compact.

In some embodiments the engagement head 2 and the duct 5 could be sold separately or assembled separately. For example, prior to use a female user may insert the duct 5 into the urine discharge end 7 of the engagement head 2 to complete the assembly of the urinary aid 1. At this point the urinary aid 1 is ready for use.

In other embodiments, owing to the compact size of the engagement head 2 it could form part of a panty or a device that is worn by a female, so that when the female wishes to urinate they merely need to either lower their zip or undo their buttons of the pants and insert the duct 5 into the urine receiving end 6 into the engagement head 2.

In some embodiments the duct 5 may be connected to the engagement head 2, but stored in a compact arrangement.

The length of the duct 5 may be straight or curved or made of flexible or stiff material.

In some embodiments the tube has an expanding concertina arrangement that allows the tube to be bent so as to direct the urine flow.

In some embodiments the tube comprises a number of valves that assists in the collection of particular amounts of urine. For example, the urinary aid 1 could be used to sample urine. The use of multiple valves could assist in this by collecting the initial urine flow, which is the disposed of, so as to sample the mid-stream urine flow for analysis. A number of different valves could be used that are responsive to a collection of a certain amount of urine or that can be manually opening or closed or are of a squeezable valve sort, squeezing of the valve cause deformation of the valve allowing urine to flow through.

The tube may also incorporate bellows or a pump. The pump or bellows could be useful for developing a positive pressure to suck liquid from the liquid-expelling opening.

### 6. Use of an Urinary Aid

As stated above, prior to use the device the urinary aid 1 can either be formed as a complete device or can be assembled prior to use.

For use, the female user parts the lips of her labia minora applying the locater element 3 against the mouth of the urethral opening. In those embodiments where the conformable material 4 is formed as a layer on the locator element 3 it will be the conformable material 4 that actually engages to the user.

Alternatively, in some embodiments the conformable material 4 forms a layer merely over the outer region 9 of the locater element 3. In such an arrangement, the locater element 3 will contact the mouth of the urethral opening and be displaced inwards thereby causing a corresponding outward displacement of the outer region 9 of the locater element 3 bring the conformable material 4 that is present as a layer over the outer region 9 of the locater element 3 against the user to form a seal.

In a further embodiment the conformable material 3 may both be formed (1) on the locator element 3 and (2) radially about the locator element 3.

Shown in FIG. 12 is an example of an urinary aid 1 wherein the urinary aid 1 comprises an engagement head 2 with the conformable material 4 located radially about the locater element 3.

It is seen in FIG. 12 that upon engagement of the locater element 3 with the mouth of the urethral opening the outer region of the locater element 3 is displaced forwards causing a seal to be formed by the conformable material 4 against the user. Therefore, should any urine escape between the seal formed between the locater element 3 and the mouth of the urethral opening, the urine will be caught by the conformable material 4 which will prevent leakage.

Once the female user begins to urinate, the urine flows through the engagement head 2 and into the duct 5.

It should be appreciated that as females may have different anatomy the urinary aid can be manufactured in variety of sizes to suit. It should also be appreciated that the locater elements 3 seen in FIGS. 12 and 13 would not normally be visible, except with a tube, as it would be covered by the folds of the labia minora.

In those embodiments comprising a projection 10 as seen in FIG. 13, the projection is inserted into the urethral opening and the seal is formed about the projection.

### 7. Portability

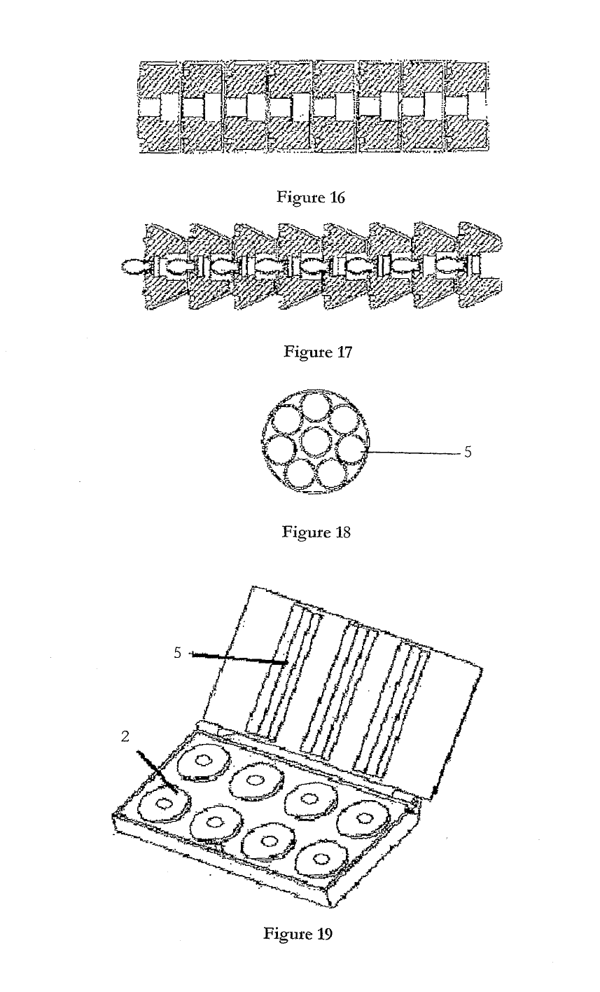

An advantage of the urinary aid 1 of the present invention is its small size and thus greater portability. As shown in FIGS. 16 and 17 are examples of how an urinary aid 1 could be index stacked for ease of portability. Such index stacking could also be used in a dispenser unit that may be present in a urinal.

In some embodiments the index stacking of the urinary aid 1 could be in a device as shown in FIG. 15. FIG. 15 shows a cylindrical pen-type carrying arrangement that can be clipped to the top of a trouser pocket or carried in a purse or handbag or other such thing. The outer body is a cylindrical body that can house the duct. The upper part of the cylindrical pen-type arrangement shown in FIG. 15 may include a top cover that is removed from prior to use. The pressure of the pen may be used to push a carriage passed through a raised ridge to bring the engagement head 2 to the top of the cylindrical pen.

The stack arrangement of the engagement head 2 could be used with a tube arrangement as shown in FIG. 18. Here the ducts 5 are arranged radially which can be used with the pen like arrangement to prepare an urinary aid 1.

For example, pushing down on a portion on the top of a pen (i.e. a ring) can engage an engagement head 2 through the insertion of a duct 5 within to the engagement head 2. The assembled urinary aid 1 can then be removed from the pen like device and utilised. The urinary aid 1 can then be thrown away. Second and subsequent urinary aids can be prepared in the same manner through pushing down on the ring that engages each subsequent engagement head 2 with a duct 5.

As seen in FIG. 19 is a pack that could be used to transport a number of engagement heads and/or tubes 5. The engagement heads can be mated with a duct at a later point prior to use.

### 8. Method of Manufacture

Another aspect of the invention relates to a method of making an urinary aid of the present invention comprising the steps (in no particular order) of:

forming the locater element, wherein the locator element is adapted to seal about the mouth of the urethra,

forming the absorbable material on or about the locator element,

attaching a duct to the locator element and absorbable material pair,

such that when in use the urine passes from the urethra past the locator element and into the duct.

In some embodiments the locator element is formed unitarily with the absorbable material. Preferably the locator element material is compressed to form it as a rigid material. More preferably the locator element and absorbable material are compression moulded, with the locator element material having greater compression (and thus increased density) compared to the absorbable material.

In an alternate embodiment the locator element and the absorbable material are formed from separate materials. Preferable the locator element and the absorbable material are bonded to each other. In one embodiment the locator element and the absorbable material are heat moulded together, the heat moulding bonding the two materials together.

In some embodiments, the urinary aid 1 is formed as an expandable blank that can be expanded to the use condition through inflation or expansion of the lie-flat blank. The advantage of such a lie-flat blank is that it will be easy to store and transport. For example, the engagement head 2 that defines the locater element 3 and the conformable material 4 may be made from material that is compressible. This compressible material would have a memory so that when flattened and released from that flattened state, to which it is biased, it will form the three-dimensional state that is the condition for use. The duct may yet form part of the device or be inserted into the bias following its transition from the flat state to the fully 3-dimensional in use state.

In one embodiment the urinary aid 1 is packaged in a compressed condition. Preferable the compressed urinary aid is biased to reform to its 3-dimensional condition.

It should be appreciated that may different methods could be used to manufacture the device the urinary aid 1 of the present invention. Such methods may use compression moulding or heat moulding if the conformable material 4 and the locater element 3 of the engagement head 2 are formed from either the same material or differing materials.

### 9. Biodegradability

In some embodiments, part or all of the urinary aid 1 is biodegradable. For example, in some embodiments the engagement head 3 is biodegradable. In alternate embodiments, the engagement head 2 and the duct 5 are both biodegradable.

In alternate embodiments, the conformable material 4 may be biodegradable as may also be the duct 5 or the locater element 3 or the shell 11.

In some embodiments at least part thereof of the urinary aid 1 is biodegradable. Preferably the locator element 3 is biodegradable. Preferably the locator element 3 and the conformable material 4 are both biodegradable. Preferably the locator element 3, the conformable material 4 and the duct 5 are all biodegradable. Preferably the conformable material 4 is biodegradable. Preferably the conformable material 4 and the duct 5 are both biodegradable. More preferably the entire urinary device 1 is biodegradable.

In summary, some features of the urinary aid 1 of the present invention include:

The entire urinary aid 1 is placed within the folds of labia minora, and seals against the raised circular portion of the urethra opening. Therefore, the urinary aid 1 does not need to take its location from any other feature of the females anatomy, such as the clitoris or vagina. Nor does the urinary aid 1 cover any part of a female's anatomy outside the folds or lips of the labia minora.

The urinary aid 1 uses a conformable material 4, that preferably is absorbent, that forms a seal around the urethra opening as it compresses to the shape of an individual's anatomy. This removes any need to press a cup shape item over the urethra opening using a shield or a covering.

The urinary aid 1 uses a locator element 3 that locates over the raised portion surrounding the urethra opening. The locator element 3 is moveable allowing it to “self-align” with the urethral opening, helping to form a seal.

As the urinary aid 1 does not allow leakage of urine, it substantially reduces the risk of infections incurring in the vaginal passage from cross-contamination with urine

The urinary aid 1 can be disposed off after a single use.

The urinary aid 1 covers only the region around the urethra opening, resulting in a small compact size. For example, in one embodiment the overall size is about 20 mm in diameter and about 60 mm in length. This makes it very portable and discreet.

### Example

The urinary aid 1 of this example is compact and portable. It enables women to urinate like a male. The urinary aid 1 is held against the female's urethra opening, within the folds of labia minora, without covering the vaginal passage opening or the pubic area. The urinary aid is made available in standard sizes, without the need to be personalised.

The urinary aid consists of a locator element 3 that locates the urinary aid 1 to the urethral opening to direct the flow of urine away from the urethral opening with minimal or no leakage to the surrounding area. Two types of urinary aids are available. The first urinary aid is a modified nozzle that forms a contoured face circular seal when pressed around the mouth of urethra. The forward end sits away from the urethra and has a circular opening of comparatively generous section through which the urine flows. The second aid has a smooth rounded edge hollow nozzle that is small enough to be inserted into the urethral opening. The forward end sits away from the urethra and has a circular opening of comparatively generous section through which the urine flows.

The urinary aid also includes an absorbing seal element that is made of a highly absorbent material. This absorbing seal element is about 5 mm in thickness and forms a tight surface contact seal with the wall of labia minora so as to absorb any leakage that may escape past the locator element. The locator element, whether the smooth rounded edge nozzle or the profiled circular seal, has a circular passage that is in line with the axis of the locator element that is coated to make it liquid resistant. This ensures the urine cannot leak to the surrounding regions. The outer edge of the contoured face has a profile to suit the region surrounding the urethra opening within the labia minora. This is a bottom semicircular with a triangular top with a rounded apex to suit the triangular region of a female's anatomy within the labia minora around the urethra opening.

The part of the urinary aid that does not come into contact with the urethra is covered by a thin plastic part referred to as the shell element. The shell element is shaped to cover the surface of the absorbing seal element. The shell element has a hollow saucer-like shape, or cylindrical shallow dish-like shape, with the hollow portion facing the urethra. The shell element closely conforms to the shape of the absorbing seal element, with a circular opening located at the end opposite to the urethra through which a thin wall tube (“tube element”) is inserted.

The tube element is inserted in the absorbent seal element to direct the urine away from the user and is made of thin plastic material, or material that is coated to make the passage temporary impervious to urine. The tube may be straight or curved, with slight flexibility to direct the flow, or with a bellow-type portion that permits bending of the tube up to about 45 degrees.

Urinary aids are produced with locator elements, absorbing seal elements, shell elements and tube elements of particular sizes and profiles to account for the varying sizes and shapes of women anatomy in the urethra region. The urinary aid is also made of particular material (biologically degradable or recyclable), and with particular surface coatings to account for personal preferences regarding hygiene and comfort.

In one type of urinary aid the locator element is a smooth rounded edge short nozzle made of plastic material. For example, polycarbonate, medical grade thermo-sensitive PVC material that is a non-irritant to the delicate mucous membranes of the urethra, LDPE or plastic material that is elastic and easy to mould in thin sections, or coated paper/fabric. This type of locates element has an inserting end that is small enough to be inserted into the urethra, a slightly bulging section along the axis in the middle of the nozzle that may be circular or elliptical so as to make good contact with the walls of urethra, yet is of thin elastic section so as not to cause discomfort to the user.

Attached is a washer like rigid ring that snugly fits into a groove in the absorbing seal element that permits small movements to facilitate alignment with the urethra passage. It is generally of circular section having a comparatively larger sectional area to permit easy flow of urine.

For the locator element of a type that is inserted into the urethra, it is coated with a fine filament of lubricant to ease insertion. The lubricant is like a moisturizing body lotion an includes an anti-bacterial and perfume.

For the locator element of a type that forms a contoured face circular seal when pressed around the mouth of the urethra opening, it is connected to a washer like rigid-ring. This connection is by way of a circular passage, that is a thin tube like structure. The washer-like rigid ring fits into a groove in the absorbing seal element to permit small movements of the contoured circular face seal so that it can self align with the mouth of the urethra and also permit the flow of urine. The contoured face of the circular seal is a concave inner annular ring and convex outer annular ring, for example, somewhat like a saucer with a central hole, blended to form an effective seal with the region around urethra opening. The contoured face circular seal connected by the thin tube to the rigid ring is moulded in one piece.

In another form the contoured face circular seal is formed by having a raised annular projection on the face of the absorbing seal element to form an effective seal with the region around urethra opening. It has a central circular passage in the absorbing seal element that is coated to make the passage temporary impervious to urine.

The absorbing seal element is made of highly absorbent material such as wood pulp, or contain polyacrylate polymers (an absorbent material in powder form packed inside layer of wood pulp), and/or absorbing fabric/paper and may be coated with an anti-bacterial/antiseptic film to provide hygiene and comfort.

The shell element is formed by moulding from plastic material or chemically treated compressed fabric/paper. Another form of the shell is produced by coating the surface of the absorbing seal insert element, that is not in contact with the labia minora region, with a plastic film or lacquer or chemical treatment to form a fairly firm, impervious liquid resistant surface.

The urinary device can be assembled as one piece or in two or more sub assemblies. The subparts can easily be assembled with each other by mere insertion into one another. Such an arrangement facilitates the use of an “easy-carry” and discreet urinary aid such as, for example, a thick clip-on pen, or a compact box. These can be carried in a purse or kept in a pouch of the undergarments to provide for a female's urinary requirements for a day or so.

Where in the foregoing description reference has been made to elements or integers having known equivalents, then such equivalents are included as if they were individually set forth.

Although the invention has been described by way of example and with reference to particular embodiments, it is to be understood that modifications and/or improvements may be made without departing from the scope or spirit of the invention.

1-54. (canceled)

55. A device for sealing against a fluid opening, the device comprising a locator element that extends substantially transverse to the walls of the tube-like device, with an aperture therein to allow fluid flow, having (a) an inner region that extends from about the wall of the tube-like device head towards the centre of the tube-like device and (b) an outer region, that extends radially outwards from the wall of the tube-like device,
such that positioning of the locator element by the user against a fluid opening actuates the locator element through deflection of the inner region away from the user and simultaneous deflection of the outer region towards the fluid opening to form a seal

56. A device of claim 55 comprising a duct, wherein the duct is formed with the device, or attached prior to use.

57. A device of claim 55 wherein the locator element is a contoured circular seal adapted to form a seal when pressed around the fluid opening.

58. A device of claim 55 wherein the locater element material has a greater density and rigidity than the conformable material.

59. A device of claim 55 wherein the locator element is moveable to facilitate the sealing of the locator element to the fluid opening.

60. A device of claim 55 wherein the conformable material is present as a layer of soft absorbent material on at least part of the locator element.

61. An urinary aid able to be positioned by a female user to allow that female user to urinate with control, the device comprising:
(i) a tube-like engagement head having a urine receivable end for engaging the mouth of the urethra and a urine discharge end, tube-like engagement head comprising
(a) a locator element, located at the urine receivable end of the engagement head that allows urine from the female to access the urine discharge end of the engagement head, the locator element extending substantially transverse to the walls of the tube-like engagement head to define (a) an inner locator element region that extends from about the wall of the tube-like engagement head towards the centre of the tube-like engagement head, and (b) an outer locator element region, that extends radially outwards from the wall of the tube-like engagement head, and
(b) a conformable material attached to, or formed unitarily with, the locator element,
such that positioning of the locator element by the user against the urethral opening actuates the locator element to move into a substantially sealing configuration about the users urethral opening.

62. An urinary aid of claim 61 comprising a duct, wherein the duct is formed with the urinary device, or attached prior to use.

63. An urinary aid of claim 61 wherein positioning the locator element against the urethral opening by the user actuates the inner locator element such that the outer locator element moves towards a substantially urethral opening sealing configuration.

64. An urinary aid of claim 61 wherein the inner locator element is deflected inward of the tube-like engagement head such that the outer locator element is simultaneously deflected or moved towards or about the urethral opening of the user or into a substantially sealing configuration.

65. An urinary aid of claim 61 wherein, in use, positioning of the locator element against the urethral opening causes the locator element to act as a fulcrum as the urethral opening pushes against the central locator element region causing the proximal locator element region to move towards the female user, resulting in a seal being formed between the user and the urinary aid.

65. An urinary aid of claim 61 wherein the locator element is a contoured circular seal adapted to form a seal when pressed around the mouth of urethra opening.

67. An urinary aid of claim 61 wherein the locator element self aligns to the mouth of the urethra to form the seal.

68. An urinary aid of claim 67 wherein the locator element is moveable to facilitate the sealing of the locator element to the mouth of the urethra.

69. An urinary aid of claim 61 wherein the conformable material is present as a layer of soft absorbent material on at least part of the locator element.

70. An urinary aid of claim 69 wherein the conformable material is present on that part of the locator element that forms a seal against the user.

71. An urinary aid of claim 61 wherein the conformable material is present about the locator element.

72. An urinary aid of claim 61 wherein the locater element material has a greater density and rigidity than the conformable material.

73. An urinary aid of claim 61 wherein the locator element and the conformable material are formed from a single material.

74. An urinary aid of claim 61 wherein the locator element and the conformable material are formed from different materials.

75. An urinary aid of claim 61 wherein the urinary aid comprises one or more sealing rings exterior to said locator element.

76. An urinary aid of claim 61 wherein the seal between the user and the urinary aid is facilitated by hydration of one or both of the locator element or the conformable material.

77. An urinary aid of claim 61 wherein at least part of the urinary aid is biodegradable.

78. An urinary aid of claim 61 wherein the locator element is biodegradable.

79. An urinary aid of claim 61 wherein the locator element and the conformable material are both biodegradable.

80. An urinary aid of claim 61 wherein the locator element, the conformable material and the duct are all biodegradable.

81. An urinary aid of claim 61 wherein the conformable material is biodegradable.

82. An urinary aid of claim 61 wherein the conformable material and the duct are both biodegradable.

83. An urinary aid of claim 61 wherein the entire urinary device is biodegradable.

84. An urinary aid of claim 61 wherein the locator element and/or conformable material are coated or imbibed with a lubricant, an anti-bacterial or a perfume, or a combination thereof.

85. An urinary aid of claim 61 wherein the locator element is formed, at least in part by, wood pulp, tissue, polyacrylate polymers, absorbing fabric, absorbing paper, gelatine, starch, cellulose, alginate or carageenan.

86. An urinary aid of claim 61 wherein the absorbable material is formed, at least in part by, wood pulp, tissue, polyacrylate polymers, absorbing fabric, absorbing paper, gelatine, starch, cellulose, alginate or carageenan.

87. An urinary aid able to be positioned by a female user to assist that female to urinate with control, the device comprising or including
a duct (formed with the device or applied prior to use) to proximately receive urine and to distally discharge urine,
a first annular seal located about the proximal inlet of the duct, the first annular seal adapted to seal against the urethra and be contained within the labia minora,
a second annular seal located exterior to the first annular shield, the second annular shield adapted to seal against the user, and be contained within the labia minora.

88. An urinary aid of claim 87 wherein the seal between the first annular seal or the second annular seal and the user is facilitated by hydration.

89. An urinary aid of either claim 87 wherein the first annular seal has increased rigidity and density compared to the second annular seal.

90. An urinary aid of any claim 87 wherein the second annular seal is raised in relation to the first annular seal

91. An urinary aid of claim 87 wherein the urinary aid is biodegradable.

92. An urinary aid of claim 87 wherein urinary aid is coated or imbibed with a lubricant, an anti-bacterial or a perfume, or a combination thereof.

93. An urinary aid of claim 87 wherein the annular seals are formed, at least in part by, wood pulp, tissue, polyacrylate polymers, absorbing fabric, absorbing paper, gelatine, starch, cellulose, alginate or carageenan.

94. An urinary aid of claim 61 wherein the urinary aid comprises a shell.

95. An urinary aid of claim 61 wherein the duct comprises a one way valve.

96. An urinary aid of claim 95 wherein the valve only opens when about 1, 2, 3, 4, 5, 6, 7, 8, 9, 10, 11, 12, 13, 14, 15, 16, 17, 18, 19, 20, 25, 30, 35, 40, 45, or 50 ml of urine is collected.

97. An urinary aid claim 61 comprising an air excluder, aperture or inlet separate to the duct inlet or outlet.

98. An urinary aid of claim 61 wherein the duct is a flexible hose.

99. An urinary aid of claim 61 wherein the duct is telescopic.

100. A method of making an urinary aid of the present invention comprising the steps (in no particular order) of:
forming the locater element, wherein the locator element is a fulcrum and is adapted to seal about the mouth of the urethra,
forming the absorbable material on or about the locator element,
attaching a duct to the locator element and absorbable material pair,
such that when in use the urine passes from the urethra past the locator element and into the duct.

101. A method of claim 100 wherein the locator element is formed unitarily with the absorbable material.

102. A method of claim 100 wherein the locator element and/or absorbable material are compression moulded.

103. A method of claim 100 wherein the locator element has a density and rigidity greater than that of the absorbable material.

104. A method of claim 103 wherein the locator element and the absorbable material are formed from separate materials.

105. A method of claim 104 wherein the locator element and the absorbable material are bonded to each other.

106. A method of claim 104 wherein the locator element and the absorbable material are heat moulded together, the heat moulding bonding the two materials together.

107. A method of any one of claim 100 wherein the urinary aid comprises the step of forming the device into a lie flat blank, wherein the lie flat blank can be inflated or pulled into its 3-dimensional operational state.

108. A method of making an urinary aid of claim 61.

109. The use of an urinary aid comprising the steps of
(ii) obtaining an urinary aid that comprises
(a) a duct to proximately receive urine and to distally discharge urine,
(b) a locator element, located at the proximal inlet end region of the duct that allows urine from the female to access the duct, that effectively extends substantially transverse to the duct to define (a) a central locator element region that extends from about the wall of the duct towards the notional centre of the duct, and (b) a proximal locator element region, that extends away from the wall of the duct,
(c) a conformable and absorbable material (“conformable material”) attached or formed unitarily with the locator element,
(iii) wherein the female user applies the device to the mouth of her urethra so that the locator element forms a seal substantially about the urethral mouth, such that the locator element is positioned within the confounds of the labia minora.

110. The use of an urinary aid of claim 109 including the step whereby the female user presses the urinary aid against the mouth of the urethra so that the conformable material, located about the locator element, pushes forward in a fulcrum motion so that the conformable material forms a seal against the female user.

111. The use of an urinary aid of claim 109 wherein the seal is formed within the confounds of the labia minora.

112. The use of an urinary aid of claim 61.

113. A pack of urinary aids of claim 61, without or without the duct.

113. An urinary aid substantially as described herein, with or without reference to claim 61.
